SEQUENCE LISTING (8 sequences)

SEQ ID NO: 1 - DNA, 3635 bp, Entamoeba histolytica
gtaggcctgataaacttaatgaattttcagcagatattgattattatgaccttggtattatgtcccgtggaaagaatgcaggttcatggtatcattcttatgaacatcaatatgatgttttctattatttagctatgcaaccatggagacattttgtatggactacttgtacaacaactgatggcaataaagaatgttataaatatactatcaatgaagatcataatgtaaaggttgaagatattaataaaacagatattaaacaagatttttgtcaaaaagaatatgcatatccaattgaaaaatatgaagttgattgggacaatgttccagttgatgaacaacgaattgaaagtgtagatattaatggaaaaacttgttttaaatatgcagctaaaagaccattggcttatgtttatttaaatacaaaaatgacatatgcaacaaaaactgaagcatatgatgtttgtagaatgcatttcattggaggaagatcaattacattcagatcatttaacacagagaataaagcatttattgatcaatataatacaaacactacatcaaaatgtcttcttaaagtatatgataataatgttaatacacatcttgcaattatctttggtattactgattctacagtcattaaatcacttcaagagaatttatctcttttaagtcaactaaaacagtcaagagtaacactctactatcttaaagatgtatcttattttacagttaatattactttaaatgatttgaaatatgagacacttgtccaatacacagcaggttgaggacaagttgatccacttgttaatcaagctaagaatgacttagctactaaagttgcagataaaagtaaagataaaaatgcaaatgataaaatcaaaagaggaactatgattgtgttaatggatactgcacttggatcagaatttaatgcagaaacagaatttgatagaaagaatatttcagttcatactgttgttcttaatagaaataaagactcaaagaatacacgtagtgcattgagacttgtttcacttggaccacattatcatgaattcacaggtaatgatgaagttaatgcaacaatcactgcacttttcaaaggaattagagccaatttaacagaaagatgtgatagagataaatgttcaggtttttgtgatgcaatgaatagatgcacatgtccaatgtgttgtgagaatgattgtttctatacatcctgtgatgtagaaacaggatcatgtattccatggcctaaagctaaaccaaaagcaaagaaagaatgtccagcaacatgtgtaggtttatatgaatgtaaagatcttgaaggatgtgttgttacaaaatataacgcatcttgtgaaccaaaagtgaaatccatggtaccatattgtgataacgataagaatctaactgaagtatgtaaacaaaaagctaattgtgaagcagatcaaaaaccaagttctgatggatattgttggagttatacatgtgaccaaactactggtttttgtaagaaagataaacgtgctgaaaatatgtgtacaggaaagacaaataactgtcaagaatatgtttgtgattcagaacaaagatgtactgttcaagaaaaggtatgtgtaaaaacatcaccatacattgaaatgtcatgttatgtagctaagtgtaatctcaatacaggtatgtgtgagaacagattatcatgtgatacatactcatcatgtggtggagattctacaggatcagtatgtaaatgtgattctacaactaataaccaatgtcaatgtactcaagtaaaaaacggtaattattgtgattctaataaacatcaaatttgtgattatacaggaaaaacaccacaatgtaaagtgtctaattgtactgaagatcttgttagagatggatgtcttattaagagatgtaatgtaacaagtaaaacaacatattgggagaatgttgattgttcaaacactaagattgaatttgctcaagatggtaaatctgaaactatgtgtaaaccatattactcagctacatgtttgaatggacaatgtgttgttcaagcagttggtgatgtttctaatgtaggatctggatattgttcaatgggaacagataatattattacatatcatgatgattgtaattcacgtaaatcacaatgtggaaactttaatggtaagtgtgtagaaaatagtgacaaatcatattcttgtgtatttaataaggatgtttcttctacatcagataatgatatttgtgcaaaatgttctagtttaacatgtccagctgatactacatacagaacatatacatatgactcaaaaacaggaacatgtaaagcaactgttaagccaacaccatcatgttcagtatgcgaaaaaggtaaatttgtagagaaatcgaaagatcaaaaattagaacgtaaagtcactttagaagatggaaaagaatatcaatacaatattccaaaagattgtgtcaatgaacaatgcattccaacaagcatacatgattgtttaggtaatgatgataactttaaatctatttataacttctatttaccatgtcaagcatatgttacagctacctatcattacagttcattattcaatttaactagttataaacttcatttaccacaaagtgaagaatttatgaaagaggcagacaaagaagcatattgtacatacgaaataacaacaagagaatgtaaaacatgttcattaattgaaactagagaaaaagtacaagaagttgatttgtgtgcagaagagactaagaatggaggagttccattcaaatgtaagaataacaattgcattattgatcctaactttgattgtcaacctattgaatgtaagattcaagagattgttattacagaaaaagatggaataaaaacaacaacatgtaaaaatggtacaaaaacaacatgtgacactaacaataagagaatagaagatgcacgtaaagcattcattgaaggaaaagaaggaattgagcaagtagaatgtgcaagtactgtttgtcaaaatgataatagttgtccaattattgctgatgtagaaaaatgtaatcaaaacacagaagtagattatggatgtaaagcaatgacaggacaatgtgatggtactacatatctttgtaaatttgtacaacttactgatgatccatcattagatagtgaacattttagaactaaatcaggagttgaacttaacaatgcatgtttgaaacataaatgtgttgagagtaaaggaagtgatggaaaaatcacacataaatgggaaattgatacagaacgatcaaatattgatccaaaacaaagaaatccatgcgaaaccgcaacatgtgatcaaacaactggagaaactatttacccaatgaaaacatgtactgtttcagaagaattcccaacaatcacaccaaatcaaggaagatgtttctattgtcaatgttcatatcttgacggttcatcagttcttactatgtatggagaaacagataaagaatattatgatcttgatgcatgtggtaattgtcgtgtttggaatcagacagatagaacacaacaacttaataatcacaccgagtgtattctcgcaggagaaattaataat

SEQ ID NO: 2 - protein, 1276 aa, Entamoeba histolytica
GRLDEFSADNDYYDGGIMSRGKNAGSWYHSYTHQYDVFYYLAMQPWRHFVWTTCDKNDNTECYKYTINEDHNVKVEDINKTNIKQDFCQKEYAYPIEKYEVDWDNVPVDEQRIESVDINGKTCFKYAAKRPLAYVYLNTKMTYATKTEAYDVCRMDFIGGRSITFRSFNTENKAFIDQYNTNTTSKCLLNVYDNNVNTHLAIIFGITDSTVIKSLQENLSLLSQLKTVKGVTLYYLKDDTYFTVNITLDQLKYDTLVKYTAGTGQVDPLINIAKNDLATKVADKSKDKNANDKIKRGTMIVLMDTALGSEFNAETEFDRKNISVHTVVLNRNKDPKITRSALRLVSLGPHYHEFTGNDEVNATITALFKGIRANLTERCDRDKCSGFCDAMNRCTCPMCCENDCFYTSCDVETGSCIPWPKAKPKAKKECPATCVGSYECRDLEGCVVTKYNDTCQPKVKCNVFYCDNDKNLTEVCKQKANCEADQKPSSDGYCWSYTCDQTTGFCKKDKRGKEMCTGKTNNCQEYVCDSEQRCSVRDKVCVKTSPYIEMSCYVAKCNLNTGMCENRLSCDTYSSCGGDSTGSVCKCDSTTGNKCQCNKVKNGNYCNSKNHEICDYTGTTPQCKVSNCTEDLVRDGCLIKRCNETSKTTYWENVDCSNTKIEFAKDDKSETMCKQYYSTTCLNGKCVVQAVGDVSNVGCGYCSMGTDNIITYHDDCNSRKSQCGNFNGKCIKGSDNSYSCVFEKDKTSSKSDNDICAECSSLTCPADTTYRTYTYDSKTGTCKATVQPTPACSVCESGKFVEKCKDQKLERKVTLENGKEYKYTIPKDCVNEQCIPRTYIDCLGNDDNFKSIYNFYLPCQAYVTATYHYSSLFNLTSYKLHLPQSEEFMKEADKEAYCTYEITTRECKTCSLIETREKVQEVDLCAEETKNGGVPFKCKNNNCIIDPNFDCQPIECKIQEIVITEKDGIKTTTCKNTTKATCDTNNKRIEDARKAFIEGKEGIEQVECASTVCQNDNSCPIITDVEKCNQNTEVDYGCKAMTGECDGTTYLCKFVQLTDDPSLDSEHFRTKSGVELNNACLKYKCVESKGSDGKITHKWEIDTERSNANPKPRNPCETATCNQTTGETIYTKKTCTVSEFPTITPNQGRCFYCQCSYLDGSSVLTMYGETDKEYYDLDACGNCRVWNQTDRTQQLNNHTECILAGEINNVGAIAAATTVAAVIVAVVVALIVVSIGLFKTYQLVSSAMKNAITITNENAEYVGADNEATNAATFNG

SEQ ID NO: 3 - DNA, 30 bp, Artificial Sequence
  Description of Artificial Sequence Synthetic primer
ggaattgaattccatatgtgtgagaacaga

SEQ ID NO: 4 - DNA, 27 bp, Artificial Sequence
  Description of Artificial Sequence Synthetic primer
agaattgcctctagactattctgaaac

SEQ ID NO: 5 - DNA, 24 bp, Artificial Sequence
  Description of Artificial Sequence Synthetic primer
aaaacccgtcctcgttcggattgc

SEQ ID NO: 6 - DNA, 25 bp, Artificial Sequence
  Description of Artificial Sequence Synthetic primer
ccgcgttgtttcatcaagccttacg

SEQ ID NO: 7 - DNA, 24 bp, Artificial Sequence
  Description of Artificial Sequence Synthetic primer
ctgtagaagtcaccattgttgtgc

SEQ ID NO: 8 - DNA, 23 bp, Artificial Sequence
  Description of Artificial Sequence Synthetic primer
gactgcccacctgagagcggaca

(19) United States
(12) Patent Application Publication
(10) Pub. No.: US 2010/0278869 A1
(43) Pub. Date: Nov. 4, 2010

(54) Plant Produced Vaccine for Amebiasis

(75) Inventor: Henry Daniell, Winter Park, FL (US)

Correspondence Address: Timothy H. Van Dyke, 390 No. Orange Avenue, Suite 2500, Orlando, FL, 32801, US

(73) Assignee: University of Central Florida Research Foundation, Inc., Orlando, FL (US)

(21) Appl. No.: 12/709,711

(22) Filed: Feb. 22, 2010

Related U.S. Application Data
(63) Continuation of application No. 12/042,453, filed on Mar. 5, 2008, now abandoned.
Application No. 12/042,453 is a division of application No. 11/914,469, filed on Jan. 30, 2008.
(60) Provisional application No. 60/685,733, filed on May 27, 2005.

(51) Int. Cl. A61K 39/002; A61P 33/04

(52) U.S. Cl. 424/269.1

(57) ABSTRACT

Disclosed herein are methods of making a vaccine against Entamoeba histolytica and methods of immunizing a subject using such vaccine. Specifically exemplified are plants expressing a LecA polypeptide and plant material obtained from such plant being used as a basis for vaccination

### RELATED APPLICATIONS

This application is a divisional of U.S. Ser. No. 11/914,469 filed Nov. 15, 2007, which is a national stage filing of PCT/US06/21020 filed May 30, 2006, which claims priority to U.S. Ser. No. 60/685,733 filed May 27, 2005, which is incorporated herein in its entirety by reference.

### STATEMENT REGARDING U.S. GOVERNMENT RIGHTS

This investigation was supported in part by USDA 3611-21000-017-00D and NIH R01GM63879. The U.S. government may have rights in this application.

### BACKGROUND

Diarrheal diseases continue to be the major causes of morbidity and mortality in children in developing countries. In developed countries, microorganisms causing diarrheal diseases remain a major concern for their potential use as bioterrorism agents. Amebiasis caused by Entamoeba histolytica, an enteric protozoan parasite, ranks only second to malaria as a protozoan cause of death. The World Health Organization estimates that there are about 50 million cases of colitis and liver abscess annually and about 100,000 deaths each year from Entamoeba histolytica infection [21, 35, 16]. This infection occurs throughout the world but occurs mostly in the developing countries of Central and South America, Africa and Asia.

Entamoeba histolytica, is one of the most potent cytotoxic cells known and was named by Schaudinn in 1903 for its ability to destroy human tissues [32]. The life cycle of Entamoeba is simple with an infectious cyst and an invasive trophozoite. The infection initiates when the cyst form of the parasite is ingested with contaminated food or water [35, 39]. The infective cyst form of the parasite survives passage through the stomach and the small intestine. The cyst is resistant to gastric acidity, chlorination, and desiccation, and can survive in moist environment for several weeks. The cysteine-rich composition of the surface antigens may be important for the survival of the amebae in such harsh environment. Motile and invasive trophozoites are formed when excystation occurs in the bowel lumen. The trophozoites use the galactose and N-acetyl-D-galactosamine (Gal/GalNAc)-specific lectin to adhere to colonic mucins and thereby colonize the large intestine. Colitis results when the trophozoite penetrates the intestinal mucous layer, which acts as a barrier to invasion by inhibiting amebic adherence to the underlying epithelium and by slowing trophozoite motility. Proteolytic enzymes secreted by the trophozoite disrupt the intestinal mucus and epithelial barrier and facilitate tissue penetration. The trophozoite then kills the host epithelial and immune cells causing characteristic flask shaped ulcers. Finally, the parasite resists the host's immune response and survives to cause prolonged extra intestinal infection such as amebic liver abscesses [21]. Entamoeba histolytica utilizes multiple non-specific and specific means to evade host defenses and survive within the gut and extra intestinal sites of infection.

In most infections, the trophozoites aggregate in the intestinal mucin layer and form new cysts resulting in asymptomatic infection. The life cycle is perpetuated by the cysts excreted in the stool and by further fecal-oral spread. In some cases, however, once the intestinal epithelium is invaded, extra intestinal spread to the peritoneum, liver and other sites may follow. Patients with amebic colitis typically show several week history of cramping abdominal pain, weight loss and watery or bloody diarrhea. Approximately 80 percent of patients with amebic liver abscess develop symptoms relatively quickly (typically within two-four weeks), which include fever, cough, and a constant, dull, aching abdominal pain in the right upper quadrant or epigastrium. Associated gastrointestinal symptoms, which occur in 10-35 percent of patients, include nausea, vomiting, abdominal cramping, abdominal distention and diarrhea. Extrahepatic amebic abscesses have occasionally been described in the lung, brain and skin and presumably may result from hematogenous spread. Since amoebae only infect humans and some higher non-human primates, theoretically an anti-amebic vaccine could eradicate this disease.

Parasite recognition of the host glycoconjugates plays an important role in the pathogenesis of amebiasis. Amebic adherence and contact-dependent cytolysis of target cells is mediated by amebic galactose/N-acetyl-D-galactosamine-inhibitable adhesin [31]. The Gal/GalNAc lectin plays several important roles in the cytolytic activity of the parasite, in invasion and in resistance to lysis by complement. The Gal/GalNAc lectin is a heterodimer with disulfide linked heavy (170 kDa) and light (35/31 kDa) subunits, which are non-covalently associated with an intermediate subunit of 150 kDa [21, 35, 31, 29]. The genes encoding the heavy and light subunits are members of multigene families consisting of five to seven members. The heavy (170 kDa) subunit gene sequence contains amino-terminal 15-amino acid hydrophobic signal sequence, an extra cellular cysteine-rich domain of 1209 amino acids containing sites for N-linked glycosylation, and transmembrane and cytoplasmic domains of 26 and 41 amino acids, respectively [35]. Anti-lectin monoclonal antibodies directed against the cysteine-rich extracellular domain inhibit adhesion of Entamoeba histolytica in vitro [21]. The light subunit is encoded by multiple genes encoding isoforms with different posttranslational modifications. The 35 kDa isoform is highly glycosylated and lacks the acylglycosylphosphotidylinositol (GPI) anchor present on the 31-kDa isoform [33, 37]. The function of the 35- and 31-kDa subunits remains unclear. The carbohydrate recognition domain (CRD) was identified in the heavy subunit of the Gal/GalNAc lectin and it has been demonstrated that an adherence-inhibitory antibody response against this domain protects against amebic liver abscess in an animal model [16]. Therefore, the CRD of the Gal/GalNAc lectin is the potential target for colonization blocking vaccines and drugs. Preliminary studies have shown that the recombinant fragments of cysteine-rich region of lectin (termed “lecA”) containing the CRD of the Gal/GalNAc lectin conferred protection against amebiasis [20, 30].

Amebiasis can ideally be prevented by eradicating the fecal contamination of food and water. Huge monetary investments are however required in providing safe food and water in developing countries. Instead, an effective vaccine would be much less expensive and is a feasible goal. An effective expression system to produce the vaccine antigen and to provide the vaccine in cleaner form and at low costs is absolutely necessary.

Chloroplast genetic engineering offers several unique advantages which include high expression levels, low cost of production, the ability to carry out post-translational modifications and maternal inheritance of the transgenes expressed [4, 18, 8]. In addition to maternal inheritance, new failsafe mechanisms have been developed for transgene containment. For example, expression of β-ketothiolase was achieved via chloroplast genetic engineering which resulted in normal development of plants except that they were male sterile transgenic plants. This gives an advantage of gene containment in addition to maternal inheritance of the transgenes expressed via transgenic chloroplasts [38]. Also, some of the challenges faced by nuclear genetic engineering could be eliminated including position effect which is overcome by site specific integration of transgenes by homologous recombination [10, 18]. The gene silencing both at transcriptional and translational level has not been observed in transgenic chloroplasts even when expressed at very high levels of translation, up to 46.1% tsp [12] or transcription, 169-fold higher rate than nuclear transgenic plants [26]. Transcript analyses conducted on chloroplast transgenic lines showed that the engineered multigenic operons were transcribed mostly as polycistrons and were efficiently translated not requiring monocistrons for translation [36].

Expressing vaccine antigens via the chloroplast genome has proven to be advantageous as the subunit vaccines are not toxic even when expressed at high levels. Bacterial genes have high AT content and this allows for their high expression in the chloroplast; and oral delivery of vaccines yields high mucosal IgA titers along with high systemic IgG titers, enabling the immune system to fight against germs at their portals of entry. Vaccines that have already been expressed in the chloroplast include the Cholera toxin B-subunit (CTB), which does not contain the toxic component that is in CTA [10], the F1˜V fusion antigen for plague 41], the 2L21 peptide from the Canine Parvovirus (CPV) [34], Anthrax Protective antigen (PA) [43], NS3 protein as vaccine antigen for hepatitis C [2], C terminus of Clostridium tetani (TetC) [42]. Cytotoxity measurements in macrophage lysis assays showed that chloroplast-derived anthrax protective antigen was equal in potency to PA produced in B. anthracis [43]. Subcutaneous immunization of mice with partially purified chloroplast-derived or B. anthracis-derived PA with adjuvant yielded IgG titers up to 1:320,000 and both groups of mice survived (100%) challenge with lethal doses of toxin. It was reported that an average yield of about 150 mg of PA per plant should produce 360 million doses of a purified vaccine free of bacterial toxins EF and LF from one acre of land [22].

Using the chloroplast transformation, tobacco has been used for hyper-expression of vaccine antigens and production of valuable therapeutic proteins like human elastin-derived polymers for various biomedical applications [19], Human therapeutic proteins, including human serum albumin [17], magainin, a broad spectrum topical agent, systemic antibiotic, wound healing stimulant and a potential anticancer agent [13], interferon [7] and insulin-like growth factor [3] have been expressed. Several other laboratories have expressed other therapeutic proteins, including human somatotropin [40] and interferon-GUS fusion proteins [27] in transgenic chloroplasts. Also, transformation of non-green tissue plastids like cotton, soybean and carrot were recently achieved [9, 24, 25]. Carrot transformation especially opens the doors for oral delivery of vaccine antigens.

### BRIEF DESCRIPTION OF THE DRAWINGS

FIG. 1. Schematic representation of pLD-SC: The pLD-SC tobacco transformation vector has the trnI and trnA genes as flanking sequences for homologous recombination. The constitutive 16S rRNA promoter regulates the expression of aadA gene (aminoglycoside 3′ adenlyltransferase) that confers resistance to spectinomycin-streptomycin and the gene10-LecA gene encoding the Entamoeba histolytica lectin antigen. Upstream to the trnA, the vector contains the 3′UTR which is a transcript stabilizer derived from the chloroplast psbA gene.

FIG. 2. PCR analysis of Wild type and putative transformants of pLD-gene10-LecA. A) Primers land within the native chloroplast genome (3P) or the aadA gene (3M) to yield a 1.65 kb product and 5P/2M primers yield a 3.3 kb product. B) Lane 1:1 kb plus ladder, Lane 2: Positive control (Interferon clone), Lane 3-7: Transgenic lines pLD-gene10-LecA (2, 6, 8*, 14, 17), Lane 8: Negative control (Wild type). C) Lane 1:1 kb plus DNA ladder, Lane 2: Positive control (pLD-gene10-LecA plasmid), Lanes 3-7: Transgenic lines pLD-gene10-LecA (2, 6, 8*, 14, 17), Lane 8: Negative control (Wild type).

FIG. 3. Southern Blot analysis of pLD-gene10-LecA. Schematic diagram of the products expected from digestions of A) Wild type untransformed plants B) Plants transformed with pLD-SC. C) Southern blot with the flanking sequence probe of pLD-gene10-LecA transgenic plants showing homoplasmy. Lane 1:1 kb plus DNA ladder, Lane 2: Wild type, Lanes 3-6: pLD-SC transgenic lines (8*, 17) D) LecA gene specific probe showing the presence of LecA in the transgenic plants. Lane 1: 1 kb plus DNA ladder, Lane 2: Wild type, Lanes 3-6: pLD-SC transgenic lines (8*, 17).

FIG. 4. Immunoblot analysis of crude plant extracts expressing LecA. Lane 1: T₁ generation transgenic plant, Lanes 2& 4: Tₒ generation transgenic plant (28 ug of crude plant extract was loaded), Lane 6: Wild type, Lane 7: Standard protein (1 ug), Lane 9: Marker, Lanes 3, 5, 8, 10: Empty.

FIG. 5. Quantification of LecA expression levels in transgenic plants (To generation). A) Expression levels in % TSP of LecA in Young, Mature and Old leaves under regular illumination conditions (16 hr light and 8 hr dark period). B) Amount of LecA (in mg) obtained from each of the Young, Mature and Old leaves based on the fresh weight. C) Amount of LecA (in ug) obtained per mg of the leaves.

FIG. 6: Comparison of immune responses in serum samples of mice administered subcutaneously with (1) plant leaf crude extract expressing lectin with adjuvant showing mean titers of 1:9600 (2) Plant leaf crude extract expressing lectin with no adjuvant showing mean titers of 1:3600 (3) Wild type plant leaf crude extract with no immune titers.

FIG. 7 shows a polynucleotide sequence encoding a heavy subunit of the Gal/GalNAc lectin, SEQ ID NO. 1, and a polypeptide sequence of LecA, SEQ ID No. 2, which contains the CRD.

### DETAILED DESCRIPTION

The inventors successfully demonstrate expression of LecA, a surface antigen of Entamoeba histolytica, in transgenic chloroplasts and also evaluation of immunogenecity of the vaccine antigen. This is the first report of LecA expression in any cellular compartment of transgenic plants.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

Methods, vectors, and compositions for transforming plants and plant cells are taught for example in WO 01/72959; WO 03/057834; and WO 04/005467. WO 01/64023 discusses use of marker free gene constructs.

Proteins expressed in accord with certain embodiments taught herein may be used in vivo by administration to a subject, human or animalin a variety of ways. The pharmaceutical compositions may be administered orally or parenterally, i.e., subcutaneously, intramuscularly or intravenously. Thus, this invention provides compositions for parenteral administration which comprise a solution of the fusion protein (or derivative thereof) or a cocktail thereof dissolved in an acceptable carrier, preferably an aqueous carrier. A variety of aqueous carriers can be used, e.g., water, buffered water, 0.4% saline, 0.3% glycerine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of fusion protein (or portion thereof) in these formulations can vary widely depending on the specific amino acid sequence of the subject proteins and the desired biological activity, e.g., from less than about 0.5%, usually at or at least about 1% to as much as 15 or 20% by weight and will be selected primarily based on fluid volumes, viscosities, etc., in accordance with the particular mode of administration selected.

Oral vaccines produced by embodiments of the present invention can be administrated by the consumption of the foodstuff that has been manufactured with the transgenic plant producing the antigenic like particles. The edible part of the plant is used as a dietary component while the vaccine is administrated in the process.

Thus, in one embodiment, a vaccine pertains to an administratable vaccine composition that comprises an antigen having been expressed by a plant and a plant remnant. A plant remnant may include one or more molecules (such as, but not limited to, proteins and fragments thereof, minerals, nucleotides and fragments thereof, plant structural components, etc.) derived from the plant in which the antigen was expressed. Accordingly, a vaccine pertaining to whole plant material (e.g., whole or portions of plant leafs, stems, fruit, etc.) or crude plant extract would certainly contain a high concentration of plant remnants, as well as a composition comprising purified antigen that and one or more detectable plant remnant.

To evaluate the antigenicity of the expressed antigens, the level of immunoglobulin A in feces or immunoglobulin G in serum is measured, respectively, after test animals has been immunized with the antigen embodiments of the present invention by oral administration or peritoneal injection. The ability to elicit the antibody formation is measured by Enzyme-linked immunosorbent assay. In addition, the direct consumption of the transgenic plant producing the antigen induces the formation of antibodies against the specific antigen.

The vaccines of certain embodiments of the present invention may be formulated with a pharmaceutical vehicle or diluent for oral, intravenous, subcutaneous, intranasal, intrabronchial or rectal administration. The pharmaceutical composition can be formulated in a classical manner using solid or liquid vehicles, diluents and additives appropriate to the desired mode of administration. Orally, the composition can be administered in the form of tablets, capsules, granules, powders and the like with at least one vehicle, e.g., starch, calcium carbonate, sucrose, lactose, gelatin, etc. The preparation may also be emulsified. The active immunogenic ingredient is often mixed with excipients which are pharmaceutically acceptable and compatible with the active ingredient. Suitable excipients are, e.g., water, saline, dextrose, glycerol, ethanol or the like and combination thereof. In addition, if desired, the vaccine may contain minor amounts of auxiliary substances such as wetting or emulsifying agents, pH buffering agents, or adjuvants which enhance the effectiveness of the vaccines. The preparation for parental administration includes sterilized water, suspension, emulsion, and suppositories. For the emulsifying agents, propylene glycol, polyethylene glycol, olive oil, ethyloleate, etc. may be used. For suppositories, traditional binders and carriers may include polyalkene glycol, triglyceride, witepsol, macrogol, tween 61, cocoa butter, glycerogelatin, etc. In addition, pharmaceutical grades of mannitol, lactose, starch, magnesium stearate, sodium saccharine, cellulose, magnesium carbonate and the like can be used as excipients.

Antigen(s) may be administered by the consumption of the foodstuff that has been manufactured with the transgenic plant and the edible part of the plant expressing the antigen is used directly as a dietary component while the vaccine is administrated in the process.

The vaccine may be provided with the juice of the transgenic plants for the convenience of administration. For said purpose, the plants to be transformed are preferably selected from the edible plants consisting of tomato, carrot and apple, which are consumed usually in the form of juice.

The vaccination will normally be taken at from two to twelve week intervals, more usually from three to hive week intervals. Periodic boosters at intervals of 1-5 years, usually three years, will be desirable to maintain protective levels of the antibodies. It will be desirable to have administrations of the vaccine in a dosage range of the active ingredients of about 100-500 μg/kg, preferably 200-400 μg/kg. Parasite Immunology, 2003, 25, 55-58 is cited to for information on Entamoeba related vaccines.

Those skilled in the art will appreciate that active variants of the genes specifically disclosed herein may be employed to produce plant derived vaccines. J Exp Med. 1997 May 19; 185(10):1793-801 provides some specific examples of fragments of known antigenic proteins and genes coding therefor.

According to one embodiment, the subject invention relates to a vaccine derived from a plant transformed to express antigenic proteins capable of producing an immune response in a subject (human or non-human animal).

According to another embodiment, the subject invention pertains to a transformed chloroplast genome that has been transformed with a vector comprising a heterologous gene that expresses a peptide antigenic for Entamoeba histolytica. In a related embodiment, the subject invention pertains to a plant comprising at least one cell transformed to express a peptide antigenic for Entamoeba histolytica.

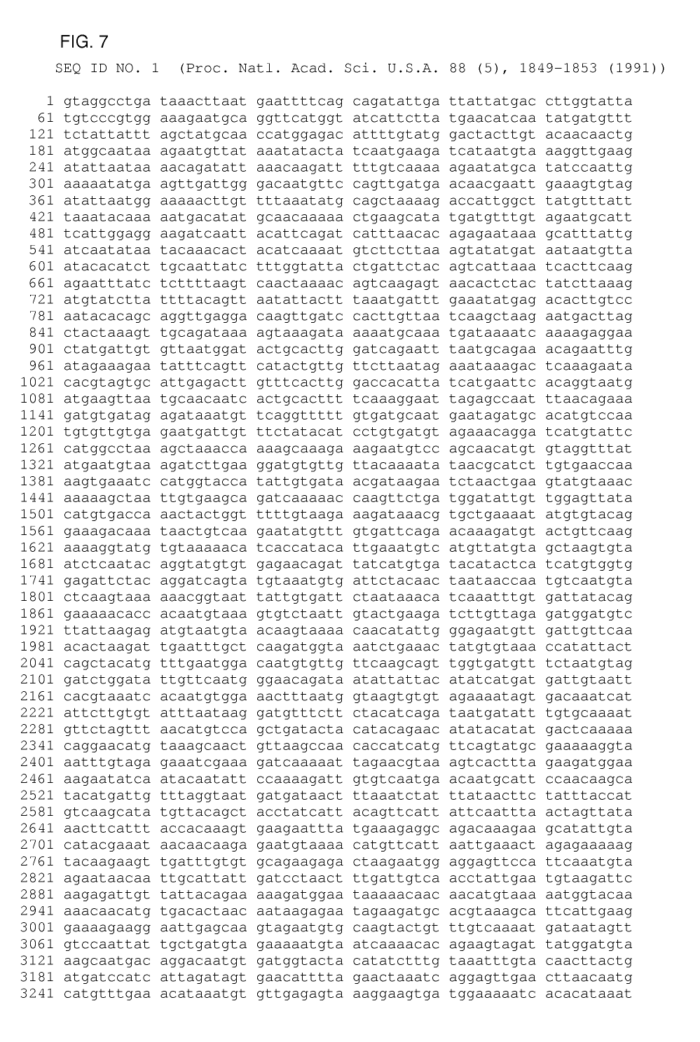

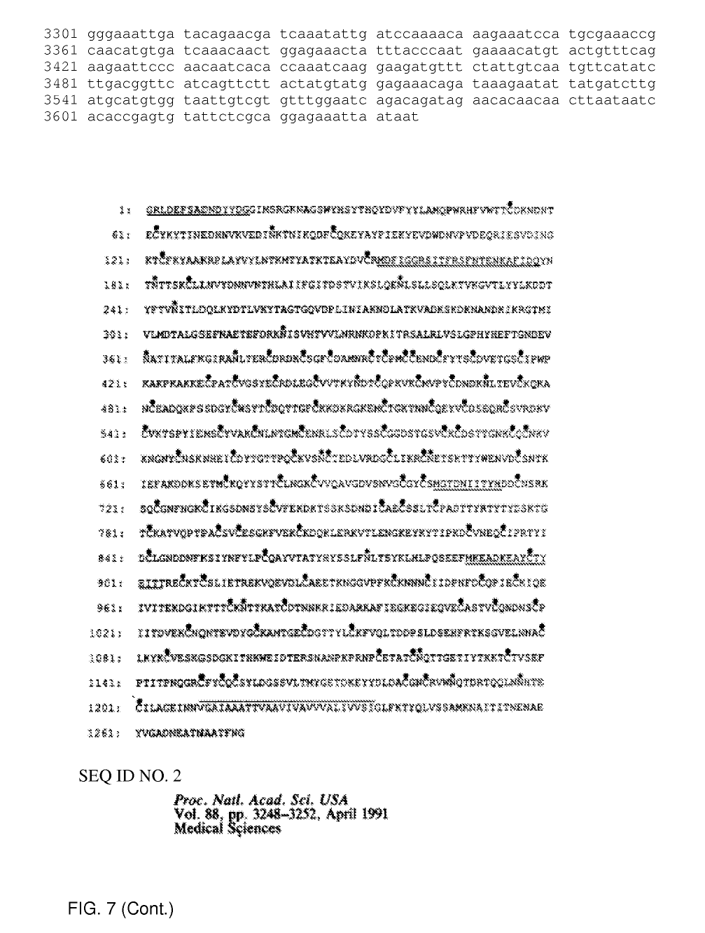

LecA polypeptides according to the invention comprise at least 12, 15, 25, 50, 75, 100, 125, 150, 175, 200, 225, 250 or 265 contiguous amino acids selected from the amino acid sequence shown in SEQ ID NO: 2, (see FIG. 7) or a biologically active variant thereof, as defined below. A LecA polypeptide of the invention therefore can be a portion of an LecA protein, a full-length LecA protein, or a fusion protein comprising all or a portion of LecA protein.

LecA polypeptide variants which are biologically active, i.e., confer an ability to induce serum antibodies which protect against infection with Entamoeba histolytica, also are considered LecA polypeptides for purposes of this application. Preferably, naturally or non-naturally occurring LecA polypeptide variants have amino acid sequences which are at least about 55, 60, 65, or 70, preferably about 75, 80, 85, 90, 96, 96, or 98% identical to the amino acid sequence shown in SEQ ID NO: 2 or a fragment thereof. Percent identity between a putative LecA polypeptide variant and an amino acid sequence of SEQ ID NO: 2 is determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of an LecA polypeptide can be found using computer programs well known in the art, such as DNASTAR software. Whether an amino acid change results in a biologically active LecA polypeptide can readily be determined by assaying for LecA activity, as described for example, in the specific Examples, below.

An LecA polynucleotide can be single- or double-stranded and comprises a coding sequence or the complement of a coding sequence for an LecA polypeptide. A coding sequence for LecA polypeptide of SEQ ID NO: 2 is shown in SEQ ID NO: 1.

Degenerate nucleotide sequences encoding LecA polypeptides, as well as homologous nucleotide sequences which are at least about 50, 55, 60, 65, 60, preferably about 75, 90, 96, or 98% identical to the nucleotide sequence shown in SEQ ID NO: 1 also are LecA polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of LecA polynucleotides which encode biologically active LecA polypeptides also are LecA polynucleotides.

Variants and homologs of the LecA polynucleotides described above also are LecA polynucleotides. Typically, homologous LecA polynucleotide sequences can be identified by hybridization of candidate polynucleotides to known LecA polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of the LecA polynucleotides disclosed herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Variants of LecA polynucleotides or polynucleotides of other species can therefore be identified by hybridizing a putative homologous LecA polynucleotide with a polynucleotide having a nucleotide sequence of SEQ ID NO: 1 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to LecA polynucleotides or their complements following stringent hybridization and/or wash conditions also are LecA polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between an LecA polynucleotide having a nucleotide sequence shown in SEQ ID NO: 1 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/1),

where 1=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

### EXAMPLES

### Materials and Methods

Construction of Vectors for Transformation of Tobacco Chloroplasts

The plasmid pcDNA 3.1 with LecA gene provided by Dr. Barbara Mann (University of Virginia Health System, Charlottesville, Va.) was used as the template to introduce start and stop codons at the N-terminal and C-terminal of the LecA gene. The primers used were forward 5′-GGAATTGAATTCC ATAT GTGTGAGAACAGA-3′ and reverse 5′-AGAATTGCCTCTAGACTATT CTG AAAC-3', respectively. The PCR amplified product of approximately 1.7 kb containing Nde I restriction site the 5′ end and Xba I at the 3′ end is obtained. The PCR product was purified using PCR purification kit (Qiagen) and was subcloned into the TOPO vector, pCR2.1-LecA. The PCR product was digested from pCR2.1-LecA vector with NdeI and NotI enzymes and subcloned into p-bluescript containing gene10 T7 bacteriophage UTR, designated pBS-g10-LecA. The final product containing the gene10 and the LecA gene (˜1.8 kb) was digested with HincII and NotI enzymes and subcloned into tobacco universal vector pLD-Ctv between EcoRV and NotI sites.

Bombardment and Selection of Transgenic Shoots

Nicotiana tabacum var. Petit havana leaves were bombarded using the Bio-Rad PDS-1000/He device. The leaves, after two days incubation period, were transferred to RMOP medium containing 500 μg/ml of spectinomycin [5, 23]. After four to six weeks, the shoots that appeared were cut in 5 mm² pieces and transferred to fresh RMOP plus spectinomycin for the second round of selection. Finally, after 4 weeks on secondary selection, the shoots were transferred to jars that contained MSO medium with 500 μg/ml spectinomycin [5, 23].

Confirmation of Transgene Integration into the Chloroplast Genome

To confirm the transgene cassette integration into the chloroplast genome, PCR was performed using the primer pairs 3P (5′-AAAACCCGTCCTCGTT CGGATTGC-3′)-3M (5′-CCGCGTTGTTTCATCAAGCCTTACG-3′) and to confirm the integration of gene of interest PCR was performed using primer pairs 5P (5′-CTGTAGAAGTCACCATTGTTGTGC-3′) and 2M (5′-GACTGCCCACCTGAG AGC-GGACA-3′) [12]. Positive control (known transgenic plant DNA sample) and Negative control (Wild type Petit havana DNA sample) were used to monitor the PCR reaction. For a 50 ul reaction volume, the PCR was set as follows: 150 ng of plant DNA, 5 μl of 10× buffer, 4 μl of 2.5 mM dNTP, 1 μl of each primer from the stock, 0.5 μl Taq DNA polymerase and H₂O to make up the total volume. The amplification was carried during 30 cycles with the following program: 94° C. for 30 sec, 65° C. for 30 sec, and 72° C. for 30 sec for the 3P-3M primer pair and 72° C. for 1 min for the 5P-2M primer pair. Cycles were preceded by denaturation for 5 min at 94° C. and followed by a final extension for 7 min at 72° C. The PCR product was analyzed on 0.8% agarose gel.

Southern Blot Analysis

The total plant DNA was extracted from transgenic T₀ plants as well as from untransformed tobacco plants using Qiagen DNeasy Plant Mini Kit. The total plant DNA was digested with HincII and run on a 0.7% agarose gel for 2.5 hours at 50 volts. The gel was then depurinated by immersing it in 0.25M HCl (depurination solution) for 15 minutes. Following, the gel was washed 2× in dH₂O for 5 minutes, and then equilibrated in transfer buffer (0.4N NaOH, 1M NaCl) for 20 minutes and then transferred overnight to nylon membrane. The membrane was washed in 2×SSC (3M NaCl, 0.3M Na Citrate) for 5 minutes, dried and cross-linked using the Bio-Rad GS Gene Cross Linker at setting C3 (150 m joules). The flanking sequence probe was obtained from the pUC-Ct vector by digesting with BamHI and BglII to obtain a 0.81 kb fragment. The gene specific probe of 400 by length was obtained by digesting pLD-SC with Bgl II and PvuII. The probes were prepared by the random primed ³²P-labeling (Ready-to-go DNA labeling beads, Amersham Pharmacia). The probes were hybridized to the membrane using Stratagene Quick-hyb solution (Stratagene, CA). The membrane was washed twice with 50 ml of wash solution (2×SSC and 0.1% SDS) at room temperature for 15 minutes. This was followed by a second round of washes with 50 ml of wash solution (0.1×SSC and 0.1% SDS) for 15 minutes at 60° C. to increase the stringency. The radio labeled blots were exposed to x-ray films and then developed in the x-ray film processor.

Western Blot Analysis:

Protein was extracted from 100 mg of plant leaf tissue both from untransformed and transformed plants and ground into fine powder with liquid nitrogen. Two hundred μl of extraction buffer (100 mM NaCl, 10 mM EDTA, 200 mM Tris-HCl-pH8, 0.05% Tween-20, 0.1% SDS, 14 mM BME, 400 mM sucrose, 2 mM PMSF) was added and the samples were mixed for 3 minutes with a micro pestle. The samples were centrifuged at 13,000×g, for 5 min to obtain the supernatant containing the soluble proteins, mixed with sample loading buffer containing BME, boiled for 5 minutes and loaded into 10% SDS-PAGE gel. The separated proteins were transferred onto a 0.2 μm Trans-Blot nitrocellulose membrane (Bio-Rad) by electro blotting in Mini-Transfer Blot Module at 85V for 45 minutes in Transfer buffer (360 ml of 10× Electrode buffer, 360 ml of methanol, 0.18 gm of SDS, 1080 ml distilled dH₂O). The membrane was blocked for one hour in P-T-M (PBS [12 mM Na₂HPO₄, 3.0 mM NaH₂PO₄—H₂O, 145 mM NaCl, pH 7.2], 0.5% Tween 20, and 3% Dry Milk) followed by transfer to P-T-M containing goat anti-lecA antibody. Membranes were then washed with distilled water and transferred to P-T-M containing rabbit derived anti-goat IgG antibody conjugated with Horseradish peroxidase (Sigma, St. Louis, Mo.). Blots were washed three times with PBST for 15 minutes each time. Then washed with PBS for 10 minutes, followed by addition of chemiluminiscent substrate ((Pierce, Rockford, Ill.) for HRP and incubated at room temperature for 5 min for the development of chemiluminescence. X-ray films were exposed to chemiluminescence and were developed in the film processor to visualize the bands.

Estimation of Total Soluble Protein

The Bradford assay was used to determine the total protein from the plant extracts. To 100 mg of ground leaf tissue from transformed and untransformed plants extraction buffer (15 mM Na₂CO₃, 35 mM NaHCO₃, 0.2 g NaN3, 0.1% Tween 20, and 5 mM PMSF adjusted to pH 9.6) was added and leaf material was ground to resuspend the proteins. Also, the extraction buffer was used to make Bovine Serum Albumin (BSA) standards ranging from 0.05 to 0.5 μg/μl. Plant extracts were diluted 1:10 and 1:20 with extraction buffer. Ten μl of each standard and 10 μl of each plant dilution was added to the wells of a 96 well micro titer plate (Cell star) in duplicates. Bradford reagent (Biorad protein assay) was diluted 1:4 with distilled water as specified and 200 μl was added to each well. Absorbance was read at 630 nm. Comparison of the absorbance to known amounts of BSA to that of the samples was used to estimate the amount of total protein.

ELISA

The quantification of LecA in the plant crude extract was done using the enzyme linked immunosorbent assay (ELISA). Transgenic leaf samples (100 mg, young, mature, old) and the wild type leaf samples (young, mature, old) were collected. The leaf samples collected from plants exposed to regular lighting pattern (16 h light and 8 h dark) were finely ground in liquid nitrogen, followed by extracting the protein from the plant leaf by plant protein extraction buffer (15 mM Na₂CO₃, 35 mM NaHCO₃, 3 mM NaN₃, pH 9.6, 0.1% Tween, and 5 mM PMSF). The mechanical pestle was used for grinding. In order to quantify the protein concentration, the standards, test samples and antibody were diluted in the coating buffer (15 mM Na₂CO₃, 35 mM NaHCO₃, 3 mM NaN₃, pH 9.6). The standards ranging from 100 to 1000 ng/ml were made by diluting purified LecA in coating buffer. The standards and protein samples (100 μl) were coated to 96-well polyvinyl chloride micro titer plate (Cell star) for 1 h at 37° C. followed by 3 washes with PBST and 2 washes with water. Blocking was done with 3% fat-free milk in PBS and 0.1% Tween and incubated for 1 h followed by washing. The primary goat anti-LecA antibody (provided by Dr. Mann, Univ. of Virginia) diluted (1:2000) in PBST containing milk powder was loaded into wells and incubated for 1 h followed by washing steps and then again incubated with 100 μl of anti-goat IgG-HRP conjugated antibody made in rabbit (American Qualex) (1:5000) diluted in PBST containing milk powder. The plate was then incubated for 1 h at 37° C. After the incubation the plate was washed thrice with PBST and twice with water. The wells were then loaded with 100 μl of 3,3,5,5-tetramethyl benzidine (TMB from American Qualex) substrate and incubated for 10-15 min at room temperature. The reaction was terminated by adding 50 μl of 2N sulfuric acid per well and the plate was read on a plate reader (Dynex Technologies) at 450 nm.

Immunization of Mice with Plant Derived Lectin Antigen:

Three groups of five female 6-7 weeks old BALB/c mice were injected subcutaneously with plant crude extracts on days 0, 15, 30, 45. Group one mice were injected with lectin (10 ug) expressing plant crude extracts along with 50 ul of alhydrogel adjuvant. Group two mice were injected with lectin (10 ug) expressing plant crude extracts with no adjuvant. Group three mice were injected with plant crude extracts of wild type tobacco plants. Blood was drawn from the retro orbital plexus 15 days after final dose (i.e., on days 60). The blood samples were allowed to stay undisturbed for 2 h at room temperature and centrifuged at 3000 rpm for 10 min to extract the serum.

ELISA to Detect the Anti-PA IgG Antibodies in the Serum Samples

96-well microtiter ELISA plates were coated with 100 μl/well of purified E. coli derived Lectin standard obtained from at a concentration of 2.0 μg/ml in PBS, pH 7.4. The plates were stored overnight at 4° C. The serum samples from the mouse were serially diluted (1:100 to 1:20,000). Plates were incubated with 100 μl of diluted serum samples for 1 h at 37° C. followed by washing with PBS-Tween. The plates were then incubated for 1 h at 37° C. with 100 μl of HRP conjugated goat anti-mouse IgG (1:5000 dilution of 1 mg/ml stock). TMB (American Qualex) was used as the substrate and the reaction was stopped by adding 50 μl of 2 M sulfuric acid. The plates were read on a plate reader (Dynex Technologies) at 450 nm. Titer values were calculated using a cut off value equal to an absorbance difference of 0.5 between immunized and unimmunized mice.

Results:

Chloroplast Transformation Vectors

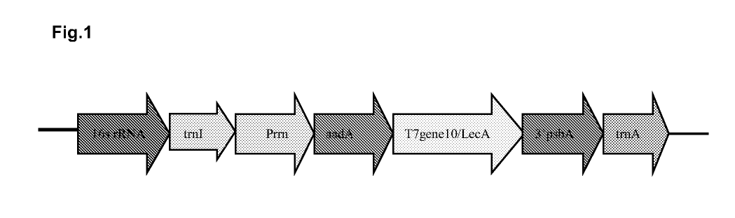

The pLD-SC vector (FIG. 1) was derived from the universal transformation vector, pLD-CtV. The pLD-SC chloroplast transformation vector containing the aadA gene, LecA coding region and 3′ psbA, integrates the transgene cassette into the trnI-trnA region of the chloroplast genome via homologous recombination. Integration of the transgene into one inverted repeat region facilitates integration into another inverted repeat via the copy correction mechanism. The psbA 3′ untranslated region (UTR) present in the transgene cassette confers transcript stability [25, 15]. The chimeric, aminoglycoside 3′ adenlyl transferase (aadA) gene, conferring resistance to spectinomycin was used as a selectable marker and its expression is driven by the 16S (Prrn) promoter [10, 5, 23]. Spectinomycin binds the 70S ribosome and inhibits translocation of peptidal tRNA's from the A site to the P site during protein synthesis. The aadA gene codes for the enzyme aminoglycoside 3′ adenlyltransferase, which transfers the adenlyl moiety of ATP to spectinomycin and inactivating it.

PCR Confirmation of Transgene Integration in Chloroplasts:

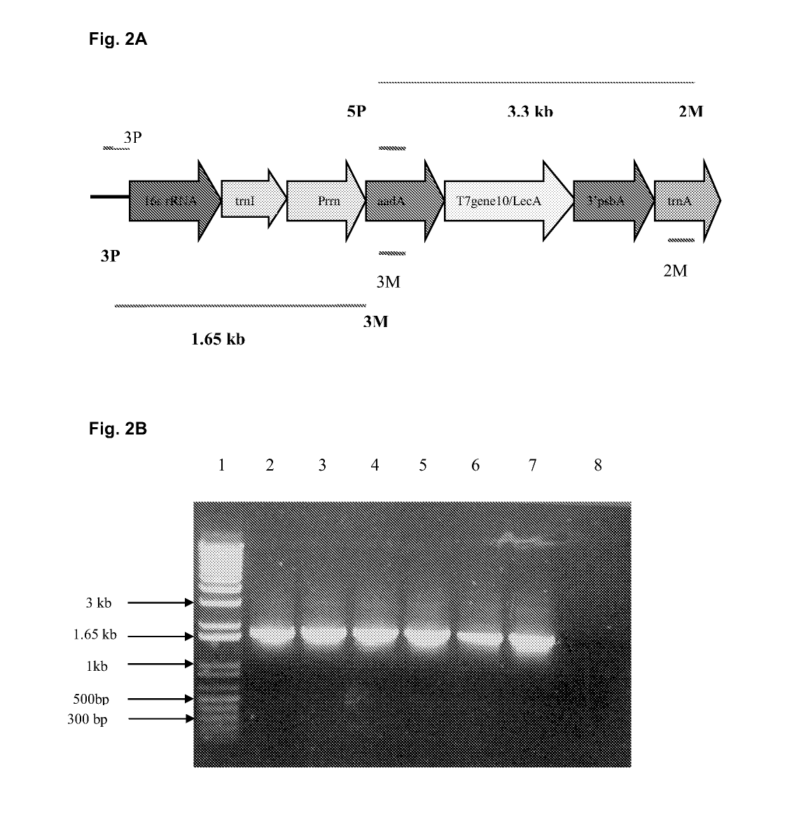

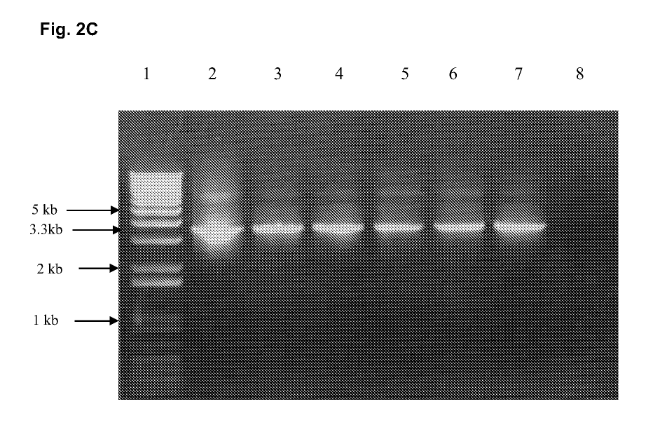

After bombardment of tobacco leaves with pLD-SC plasmid coated gold particles, about 5 shoots/plate appeared after a period of 5-6 weeks. True chloroplast transformants were distinguished from nuclear transformants and mutants by PCR. Two primers, 3P and 3M were used to test for chloroplast integration of transgenes [10, 5, 23]. 3P primer landed on the native chloroplast DNA within the 16S rRNA gene. 3M landed on the aadA gene as shown in FIG. 2A. Nuclear transformants were eliminated because 3P will not anneal and mutants were eliminated because 3M will not anneal. The 3P and 3M primers upon chloroplast integration of transgene yielded a product of 1.65 kb size fragment as shown in FIG. 2B.

The Integration of the aadA, gene10-LecA gene and 3′ psbA cassette was confirmed by using the 5P and 2M primer pair for the PCR analysis. The 5P and 2M primers annealed to the internal regions of the aadA gene and the trnA gene, respectively as shown in FIG. 2A. The product size of a positive clone is of 3.3 kb for LecA, while the mutants and the control shouldn't show any product. FIG. 2C shows the result of the 5P/2M PCR analysis. After PCR analysis using both primer pairs, the transgenic plants were subsequently transferred through different rounds of selection to obtain a mature plant and reach homoplasmy.

Achievement of Homoplasmy:

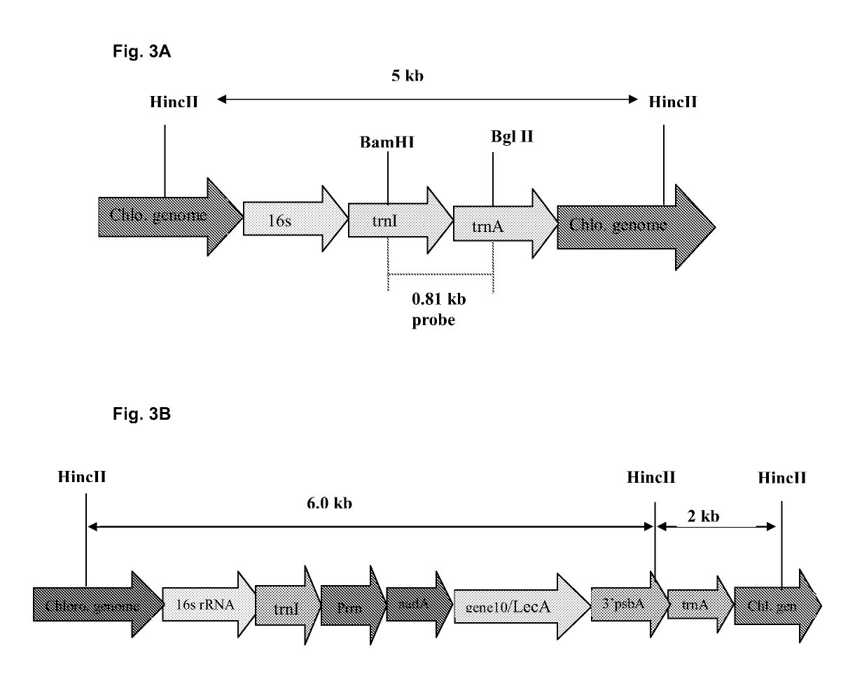

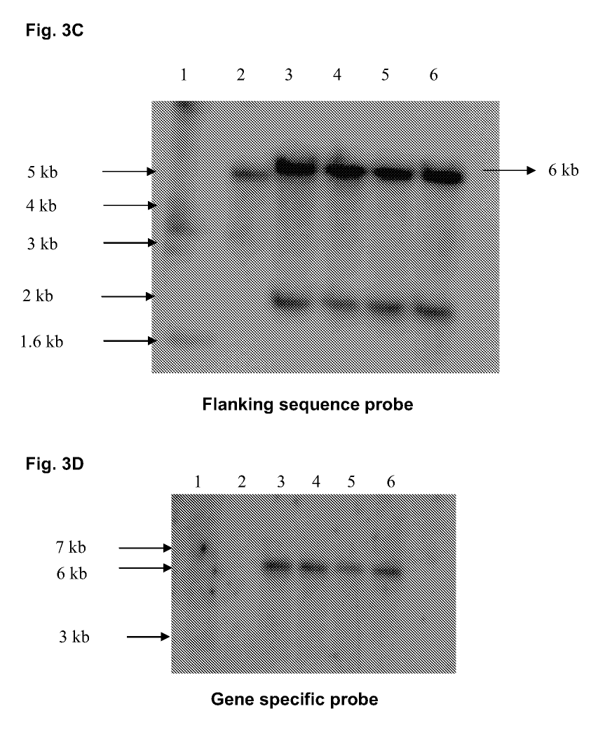

The plants that tested positive for the PCR analysis were moved through three rounds of selection and were then tested by Southern analysis for site specific integration of the transgene and homoplasmy. The DNA of the fully regenerated clones growing in jars (third selection) was extracted and used for Southern analysis. The flanking sequence probe of 0.81 kb in size allowed detection of the site-specific integration of the gene cassette into the chloroplast genome (FIG. 3A). FIG. 3B shows the HincII sites used for the restriction digestion of the plant DNA. The transformed chloroplast genome digested with HincII produced fragments of 6.0 kb and 2.0 kb for pLD-SC (FIG. 3C), while the untransformed chloroplast genome that had been digested with HincII generated a 5.0 kb fragment. The flanking sequence probe also showed if homoplasmy of the chloroplast genome has been achieved through three rounds of selection. The plants expressing LecA showed homoplasmy as there was no hybridizing wild type fragment seen in transgenic lines. The gene specific probe showed transgene integration resulting in a fragment of 6 kb as shown in FIG. 3D.

Expression of LecA in Transgenic Plants:

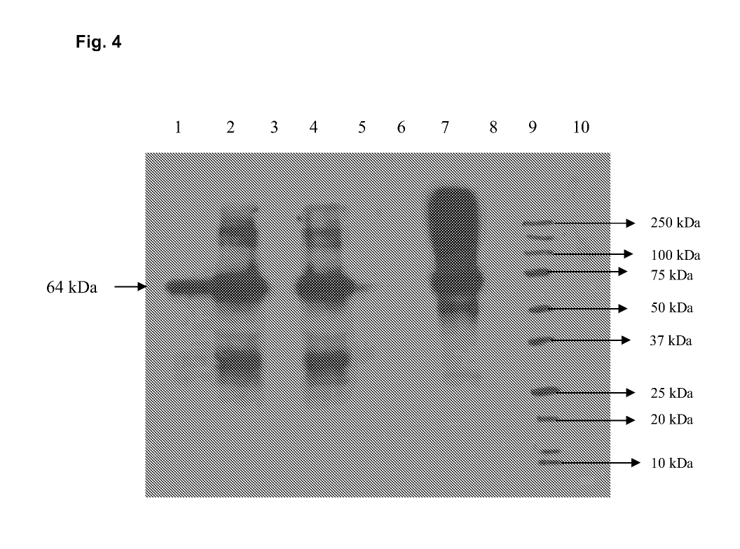

The goat anti-LecA polyclonal antibodies were used to detect the 64 kDa protein. The wild type plant (Petit havana) did not show any bands indicating that the anti-LecA antibodies did not cross react with any other proteins in the crude extract. The T1 generation plants also showed good levels of expression (FIG. 4). Each of the lanes contained around 1.5 ug of the LecA protein detected by the LecA antibodies. The lower bands seen could probably be the degraded LecA protein and the higher bands probably are the LecA protein aggregates.

Quantification of Transgenic Plant Derived LecA Protein:

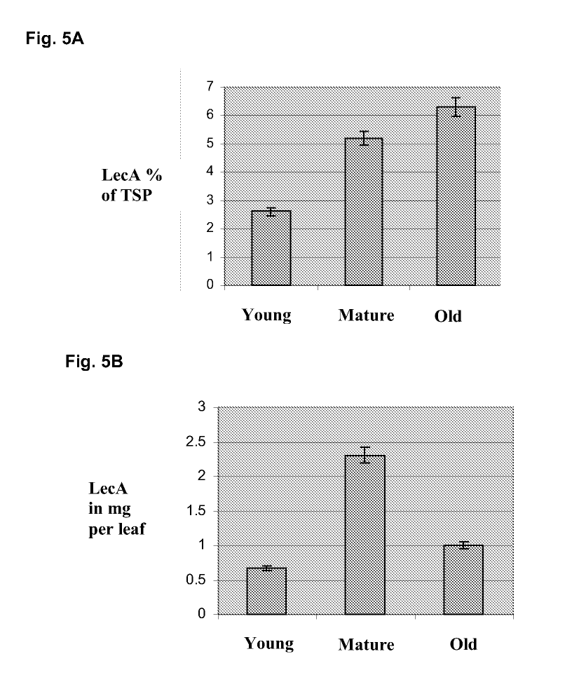

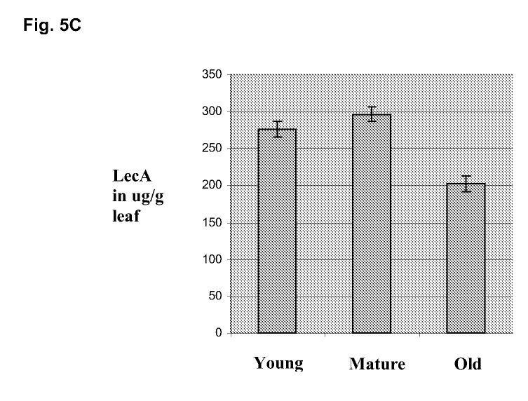

Different dilutions of purified LecA were used to obtain a standard curve. The primary antibody used was Goat polyclonal antibodies against LecA and secondary antibodies were rabbit anti-goat IgG peroxidase conjugated. The percentage of LecA expressed as a percent of total soluble protein calculated using the Bradford assay i.e. the LecA percent is inversely proportional to the TSP values. The LecA expression levels reached a maximum of 6.3% of the total soluble protein in the old leaves when compared to 2.6% TSP in young leaves and 5.2% TSP in mature leaves. The maximum LecA expression was observed in old leaves when compared to young and mature leaves (FIG. 5A). Based on the fresh weight calculations, the amount of LecA obtained from young, mature and old leaves is 0.67 mg, 2.32 mg and 1 mg per leaf respectively (FIG. 5B) and FIG. 5C shows the amount of LecA (in ug) per mg of leaf.

Evaluation of Immunogenecity:

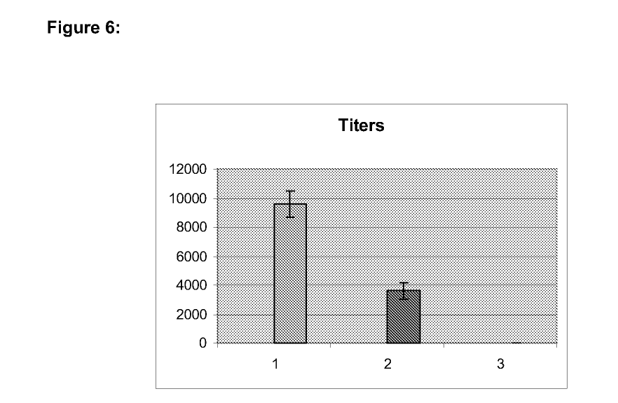

Having confirmed the expression of lectin in transgenic plants, we tested the ability of the plant derived lectin to be functional in vivo. For this the mice were immunized with crude extracts of the plant expressing lectin. The mice groups immunized with crude extracts of plant expressing lectin along with adjuvant showed immunization titers up to 1:10,000 and the mice groups immunized with plant crude extracts expressing lectin with no adjuvant showed immunization titers up to 1:4000 (FIG. 6).

Discussion:

The pLD-SC vector was derived from the universal transformation vector, pLD-CtV [5]. The pLD-SC transgene cassette is integrated into the trnI-trnA region of the chloroplast genome via homologous recombination. Expression of the LecA recombinant protein in the chloroplast depends on several factors. First, the pLD-SC vector was designed to integrate into the inverted repeat region of the chloroplast genome via homologous recombination. The copy number of the transgene is thus doubled when integrated at this site. Increased copy number results in increased transcript levels resulting in higher protein accumulation [10, 19]. Second, the T7 bacteriophage gene10 5′ UTR containing the ribosome binding site (rbs) and psbA 3′ untranslated region (UTR) used for the regulation of transgene expression help in enhancing translation of the foreign protein [15, 19]. Third, homoplasmy of the transgene is a condition where all of the chloroplast genomes contain the transgene cassette. There are 100 to 1000 chloroplasts per cell and 100 to 1000 chloroplast genomes per chloroplast [8, 12, 14]; for optimal production of the recombinant protein and transgene stability, it is essential that homoplasmy is achieved through several rounds of selection on media containing spectinomycin. If homoplasmy is not achieved, heteroplasmy could result in changes in the relative ratios of the two genomes upon cell division. The chimeric, aminoglycoside 3′ adenyl transferase (aadA) gene, conferring resistance to spectinomycin was used as a selectable marker and its expression is driven by the 16S (Prrn) promoter [10]. Fourth, expression can depend on source of the gene and its' relative AT/GC content. The prokaryotic-like chloroplast favors AT rich sequences, which reflects the respective tRNA abundance. Therefore, the LecA gene having 67% AT is expected to express well in the chloroplast. High expression of synthetic Human Somatotropin (HST), human serum albumin, human interferon-α2b, Human interferon-α, Insulin like growth factor shows that eukaryotic genes can also be expressed in the plastid [17, 7, 40, 27, 6] however; some eukaryotic genes need to be optimized for chloroplast expression. Genetic engineering of chloroplast genome to express LecA serves two purposes, high expression levels and gene containment.

PCR analysis was used to distinguish the chloroplast transformants from the nuclear transformants and the mutants. Southern blot analysis was utilized to confirm the site-specific integration of the gene cassette and also to determine the homo or heteroplasmy. High protein expression levels were obtained in the mature and old leaves of up to 6.3% of the total soluble protein, which was quantified using the enzyme linked immunosorbent assay. The difference in LecA expression levels when calculated based on percentage TSP and fresh weight is due to the low TSP in old leaves when compared to mature leaves. This could possibly be due to degradation of soluble proteins when compared to LecA. Based on fresh weight, the mature leaves showed higher expression levels as the TSP is not taken into account. More number of chloroplasts in mature leaves, large size and more number of mature leaves per plant contribute to the higher expression. An average yield of 24 mg of LecA (Table 1) per plant should produce 29 million doses of vaccine antigen per acre of transgenic plants. This shows that using plants for the production of vaccine antigens could result in low cost vaccine as compared to bacterial expression system. Differences in the titer values of the animal groups that received the extract with and without adjuvant were due to depot effect [1] and due to the alhydrogel's non-specific priming of the immune system. Control mice immunized with wild type plant leaf crude extract did not show any immune response showing the specificity of recombinant lectin elicited immune response in case of transgenic plant crude extracts.

Previous reports of vaccination with full-length native Lectin antigen in gerbils through subcutaneous route yielded titers up to 1:1024 [35]. Similarly vaccination in gerbils with 25mer peptide derived from cysteine rich region of the lectin yielded IgG titers up to 1:200 [28]. The vaccination with crude extracts of LecA transgenic lines in this study resulted in titers up to 1:10,000. This is 10-50 fold higher immunogenecity than those obtained with purified full length native lectin antigen or peptide derived from cysteine rich region of this lectin.

The vaccination with chloroplast derived LecA is much more effective in eliciting IgG antibodies than previous studies even without purification and opens a new approach for vaccine development for amebiasis. Pathogen challenge tests were not performed because BALB/c mice are not susceptible to the infection with Entamoeba histolytica. Our aim of the immunization studies in this study was to test the ability of the plant derived Lec-A to elicit the IgG immune response. The present study reports the successful expression of the LecA protein for the first time in a plant expression system.

Also, for the pathogen challenge studies, the production of IgA antibodies would be more effective over the IgG antibodies because the infection occurs predominantly in the intestinal mucosa where secretory IgA play a predominant role in effectively neutralizing the infection. Therefore, future studies involve immunization studies of orally administered plant expressing Lec-A to elicit IgA response and proceed with pathogen challenge. Development of transgenic carrot expressing LecA will open the door for the oral delivery of the vaccine and develop mucosal immune response. An ideal vaccine for Amebiasis should induce both mucosal and systemic protection. If both subcutaneous and oral delivery proves to be immunoprotective, priming both the mucosal and systemic systems may prove not only to be the cheapest way but also the most effective method of vaccination against any pathogen that attacks both the mucosal and systemic systems.

### REFERENCES

1. Audibert, F. 2003. Adjuvants for vaccines, a quest. Int. Immunopharmacol. 3:1187-1193.
2. Bhati, A. 2005. Expression of Hepatitis C viral non-structural 3 protein in transgenic chloroplasts. Master's thesis, University of Central Florida.
3. Daniell, H. 2004. Medical molecular pharming: therapeutic recombinant antibodies, biopharmaceuticals and edible vaccines in transgenic plants engineered via the chloroplast genome, p. 704-710. In R. M Goodman (ed) Encyclopedia of Plant and Crop Science, Marcel Decker, New York.
4. Daniell, H. 2002. Molecular strategies for gene containment in transgenic crops. Nat. Biotechnol. 20: 581-587.
5. Daniell, H. 1997. Transformation and foreign gene expression in plants mediated by microprojectile bombardment. Methods. Mol. Biol. 62:453-488.
6. Daniell, H., S. Chebolu, S. Kumar, M. Singleton, and R. Falconer. 2005. Chloroplast-derived vaccine antigens and other therapeutic proteins. Vaccine. 23:1779-1783.
7. Daniell, H., P. Cohill, S. Kumar, N. Dufourmantel, and M. Dubald. 2004. Chloroplast genetic engineering, p. 423-468. In H. Daniell and C. Chase (ed.), Molecular biology and biotechnology of plant organelles. Kluwer Academic Publishers, Dordrecht.
8. Daniell, H., M. Khan, and L. Allison. 2002. Milestones in chloroplast genetic engineering: an environmentally friendly era in biotechnology. Trends Plant Sci. 7: 84-91.
9. Daniell, H., S. Kumar, and N. Dufourmantel. 2005. Breakthrough in chloroplast genetic engineering of agronomically important crops. Trends Biotechnol. 23: 238-45.
10. Daniell, H., S. B. Lee, T. Panchal, and P. O. Wiebe. 2001. Expression of cholera toxin B subunit gene and assembly as functional oligomers in transgenic tobacco chloroplasts. J Mol. Biol. 311:1001-1009.
11. Daniell, H., O. N. Ruiz, and A. Dhingra. 2004. Chloroplast genetic engineering to improve agronomic traits. Methods Mol. Biol. 286: 111-137.
12. DeCosa, B., W. Moar, S. B. Lee, M. Miller, and H. Daniell. 2001. Over expression of the Btcry2Aa2 operon in chloroplasts leads to formation of insecticidal crystals. Nat. Biotechnol. 19: 71-74.
13. DeGray, G., K. Rajasekaran, F. Smith, J. Sanford, and H. Daniell. 2001. Expression of an antimicrobial peptide via the chloroplast genome to control phytopathogenic bacteria and fungi. Plant Physiol. 127:852-862.
14. Devine, A. L., and H. Daniell. 2004. Chloroplast genetic engineering for enhanced agronomic traits and expression of proteins for medical/industrial applications, p. 283-323. In S. G. Møller (ed), Plastids. Blackwell publishing, Oxford.
15. Dhingra, A., A. R. Portis, and H. Daniell. 2004 Enhanced translation of a chloroplast-expressed RbcS gene restores small subunit levels and photosynthesis in nuclear antisense RbcS plants. Proc. Natl. Acad. Sci. USA, 101: 6315-6320.
16. Dodson, J. M., P. W. Lenkowski Jr., A. C. Eubanks, T. F. G. H. Jackson, J. Napodano, D. M Lyerly, L. A. Lockhart, B. J. Mann, and W. A. Petri Jr. 1998. Infection and immunity mediated by the carbohydrate recognition domain of the Entamoeba histolytica Gal/GalNAc lectin. J Infect Dis. 179: 460-466.
17. Fernandez-San Millan, A., A. M. Mingeo-Castel, M. Miller, and H. Daniell. 2003. A chloroplast transgenic approach to hyper-express and purify human serum albumin, a protein highly susceptible to proteolytic degradation. Plant Biotechnol J. 1: 71-79.
18. Grevich, J. J. and H. Daniell. 2005. Chloroplast genetic engineering: Recent advances and future perspectives. Crit. Rev. Plant Sci. 24: 83-108.
19. Guda, C., S. B. Lee, and H. Daniell. 2000. Stable expression of biodegradable protein based polymer in tobacco chloroplasts. Plant Cell Rep. 19:257-262.

20. Houpt, E., L. Barroso, L. Lockhart, R. Wright, C. Cramer, D. Lyerly, and W. A. Petri. Jr. 2004. Prevention of intestinal amebiasis by vaccination with Entamoeba histolytica Gal/GalNAc lectin. Vaccine 22: 611-617.
21. Huston, C. D., and W. A Petri Jr. 1998. Host-pathogen interaction in Amebiasis and progress vaccine development. Eur. J. Clin. Microbiol. Infect. Dis. 17: 601-614.
22. Koya, V., M. Moayeri, S. H. Leppla, and H. Daniell. 2005. Plant based vaccine: mice immunized with chloroplast-derived anthrax protective antigen survive anthrax lethal toxin challenge. Infect. Immun. 73: 8266-8274.
23. Kumar, S, and H. Daniell. 2004. Engineering the chloroplast genome for hyper-expression of human therapeutic proteins and vaccine antigens in recombinant protein protocols. Methods Mol. Biol. 267: 365-383.
24. Kumar, S., A. Dhingra, and H. Daniell. 2004. Plastid-expressed betaine aldehyde dehydrogenase gene in carrot cultured cells, roots, and leaves confer enhanced salt tolerance. Plant Physiol. 136: 2843-2854.
25. Kumar, S., A. Dhingra, and H. Daniell. 2004. Stable transformation of the cotton plastid genome and maternal inheritance of transgenes. Plant Mol. Biol. 56: 203-216.
26. Lee, S. B., H. B. Kwon, S. J. Kwon, S. C. Park, M. J. Jeong, S. E. Han, M. O. Byun, and H. Daniell. 2003. Accumulation of trehalose within transgenic chloroplasts confers drought tolerance. Mol Breed. 11, 1-13.
27. Leelavathi, S., and V. S. Reddy. 2003. Chloroplast expression of His-tagged GUS fusions: a general strategy to overproduce and purify foreign proteins using transplastomic plants as bioreactors. Mol Breed. 11: 49-58.
28. Lotter H., F. Khajawa, S. L. Stanley Jr., and E. Tannich. 2000. Protection of gerbils from amebic liver abscess by vaccination with a 25-mer peptide derived from the Cysteine-Rich Region of Entamoeba histolytica Galactose-specific adherence lectin. Infect. Immun. 68: 4416-4421.
29. Mann, B. J. 2002. Structure and function of the Entamoeba histolytica Gal/GalNAc lectin. Intl. Rev. Cyt. 216: 59-80.
30. Mann, B. J., C. Y. Chung, J. M. Dodson, L. S. Ashley, L. L. Braga, T. L. Snodgrass. 1993. Neutralizing monoclonal antibody epitopes of the Entamoeba histolytica galactose adhesin map to the cysteine-rich extracellular domain of the 170-kilodalton subunit. Infect Immun. 61:1772-1778.
31. Mann, B. J., and L. A. Lockhart. 1998. Molecular analysis of the Gal/GalNAc adhesin of Entamoeba histolytica. J. Euk. Microbiol. 45:13 S-16S.
32. McCoy, J. J., B. J. Mann, and W. A. Petri Jr. 1994. Adherence and cytotoxicity of Entamoeba histolytica or how lectins let parasite stick around. Infect. Immun. 62: 3045-3050.
33. McCoy, J. J., B. J. Mann, T. S. Vedvick, Y. Pak, D. B. Heimark, W. A. Petri Jr. 1993. Structural analysis of the light subunit of the Entamoeba histolytica galactose-specific adherence lectin. J. Biol. Chem. 268: 24223-24231.
34. Molina, A., S. Herva-Stubbs, H. Daniell, A. M. Mingo-Castel, and J. Veramendi. 2004. High yield expression of a viral peptide animal vaccine in transgenic tobacco chloroplasts. Plant Biotechnol. J. 2:141-153.
35. Petri, W. A Jr., R. Hague, and B. J. Mann. 2002. The bittersweet interface of parasite and host: lectin-carbohydrate interactions during human invasion by the parasite Entamoeba histolytica. Annu. Rev. Microbiol. 56: 39-64.
36. Quesada-Vargas, T, O. N. Ruiz, H. Daniell. 2005. Characterization of heterologous multigene operons in transgenic chloroplasts. Transcription, processing and translation. Plant Physiol. 138: 1746-1762.
37. Ramakrishnan, G., S. Lee, B. J. Mann, and W. A. Petri Jr. 2000. Entamoeba histolytica: deletion of the GPI anchor signal sequence on the Gal/GalNAc lectin light subunit prevents its assembly into the lectin heterodimer Exp Parasitol. 96: 57-60.
38. Ruiz O. N and H. Daniell. 2005. Engineering cytoplasmic male sterility via the chloroplast genome by the expression of β-ketothiolase. Plant Physiol. 138: 1232-1246.
39. Stanley, S. L. Jr. 1997. Progress towards development of a vaccine for amebiasis. Clin. Microbiol. Rev. 10: 637-649.
40. Staub, J. M., B. Garcia, J. Graves, P. T. J. Hajduklewicz, P. Hunter, N. Nehra, V. Paradkar, M. Schlittler, J. A. Carroll, L. Spatola, D. Ward, G. Ye, and D. A. Russell. 2000. High-yield production of a human therapeutic protein in tobacco chloroplasts. Nat. Biotechnol. 18:333-338.
41. Singleton, M. L. 2003. Expression of CaF1 and LcrV as a fusion protein for development of a vaccine against Yersisnia pestis via chloroplast genetic engineering. Master's thesis, University of Central Florida.
42. Tregoning, J. S., P. Nixon, H. Kuroda, Z. Svab, S. Clare, F. Bowe, N. Fair-weather, J. Ytterberg, K. J. van Wijk, G. Dougan, and Pal Maliga. 2003. Expression of tetanus toxin fragment C in tobacco chloroplasts. Nucleic Acids Res. 31:174-1179.
43. Watson, J., V. Koya, S. Leppla, and H. Daniell. 2004. Expression of Bacillus anthracia protective antigen in transgenic chloroplasts of tobacco, a non-food/feed crop. Vaccine 22: 4374-4384.

TABLE 1
The yield of Lec A expressed in pLD-SC tobacco To transgenic lines relative to its biomass.

| | | | | Amount of | |
|---|---|---|---|---|---|
| | Leaves/ | Average weight | LecA(mg/g) | LecA (mg)/ | Amount of LecA |
| | plant | (gm) of leaf | in fresh leaf | leaf | (mg) per age group |
| Young | 3.2 | 2.5 | 0.27 | 0.67 | 2.144 |
| Mature | 7.8 | 8 | 0.29 | 2.32 | 18.096 |
| Old | 4.5 | 5 | 0.20 | 1 | 4.05 |
| Total | | | | | 24.29 |
| recombinant | | | | | |
| LecA/plant | | | | | |

Calculations: At an average yield of 24.29 mg of Lec A per plant, one acre where 8,000 plants are grown, it is 8000* 24.29 = 194,320 mg. Based on three cuttings per year, the total yield would be 582, 960 mg. With an average loss of 50% during purification, the net protein yield would be 291,480 mg. Amount of lectin for single dose of vaccine is 10 ug. At this dose, 291, 480 mg should give 29,148,000 doses or about 29 million doses.
Therefore, 29 million doses may be obtained from one acre of tobacco.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

What is claimed is:

1. A method of inducing, in a subject, serum antibodies which protect against infection with Entamoeba histolytica, comprising administering to said subject, a composition comprising a LecA polypeptide and a plant remnant.

2. A method for vaccinating a human against Entamoeba histolytica infection, comprising administering to the human an immunizing amount of a composition comprising a LecA polypeptide, wherein said LecA polypeptide is derived from a plant transformed to express said LecA polypeptide.

3. A vaccine composition comprising an immunologically effective amount of a LecA polypeptide and a plant remnant.

4. The method of claim 2, wherein said plant is transformed with a stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for comprising at least 95% identity to a SEQ ID NO. 2 (LecA protein), transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

5. The method of claim 2, wherein said LecA polypeptide is administered parenterally.

6. The method of claim 2, wherein said LecA polypeptide is administered orally.

7. The method of claim 2, wherein said plant is edible to humans.
